(19) United States
(12) Patent Application Publication
(10) Pub. No.: US 2003/0134332 A1
(43) Pub. Date: Jul. 17, 2003

(54) Diagnosis of endothelial dysfunction by nitric oxide bioactivity index

(76) Inventor: Joseph V. Boykin JR., Chester, VA (US)

Correspondence Address: BANNER & WITCOFF, 1001 G STREET N W, SUITE 1100, WASHINGTON, DC, 20001, US

(21) Appl. No.: 10/290,496

(22) Filed: Nov. 8, 2002

Related U.S. Application Data
(60) Provisional application No. 60/370,246, filed on Apr. 8, 2002.
(60) Provisional application No. 60/349,348, filed on Jan. 22, 2002.
(60) Provisional application No. 60/333,474, filed on Nov. 28, 2001.

(51) Int. Cl. G01N 33/53; C12Q 1/26; A61K 48/00; A61K 31/353; A61K 31/375; A61K 31/355

(52) U.S. Cl. 435/7.1; 435/25; 514/44; 514/456; 514/458; 514/474; 436/116

(57) ABSTRACT

Methods and kits are provided for diagnosing medical conditions of patients with a disease or condition characterized by endothelial dysfunction based on a nitric oxide bioactivity index. Nitric oxide bioactivity index is the ratio of the level of a nitric oxide-related product such as nitrate or nitrite to the level of an oxidant stress-related product such as isoprostane in plasma, urine, or another specimen from a patient. Methods are also provided for using the nitric oxide bioactivity index to treat patients with endothelial dysfunction and monitor the course of treatment.

This application incorporates by reference co-pending provisional applications Serial No. 60/333,474 filed Nov. 28, 2001, Serial No. 60/349,348 filed Jan. 22, 2002, and Serial No. 60/370,246 filed Apr. 8, 2002.

### TECHNICAL FIELD OF THE INVENTION

The invention is related to the diagnosis and treatment of medical conditions characterized by endothelial dysfunction. In particular it is related to assays for the bioactivity of nitric oxide and their use in predicting and improving clinical outcomes for patients suffering from endothelial dysfunction.

### BACKGROUND OF THE INVENTION

Reduced bioactivity of nitric oxide (NO) is a recognized feature of a form of vascular pathology termed endothelial dysfunction (P A Ashfield-Watt, S J Moat, S N Doshi, I F McDowell. Biomed Pharmacother 55, 425 (2001); V Schachinger and AM Zeiher, Coron Artery Dis 12, 435 (2001)). Endothelial dysfunction refers to abnormality in any of a number of physiological processes carried out by the endothelium, but it especially refers to abnormally low production of NO, regardless of the cause. Loss of endothelial function can adversely affect vasomotor tone (constriction and dilation), thromboregulation, inflammatory responses, and vascular smooth muscle cell growth and migration (Paterick T E, Pletcher G F. Cardiol Rev 2001 Sep-Oct;9(5):282; R Ross, N EngI J Med 340, 115 (1999); I T Meredith, A C Yeung, F F Weidinger, T J Anderson, A Uehata, T J Ryan, et. al. Circulation 87 (suppl V), V-56 (1993)). Multiple factors contribute to the genesis of endothelial dysfunction. Such factors include blood lipids, neurohumoral factors, metabolic disorders, and oxidant stress. Endothelial dysfunction results in decreased NO production by the endothelium. Loss of NO bioactivity plays a predominant role in the development of pathology in a wide variety of medical conditions (Schachinger, supra; G Cella, F Bellotto, F Tona, A Sbarai, F Mazzaro, G Motta, J Fareed, Chest 120, 1226 (2001)). Endothelial dysfunction leads to decreased vascular compliance and a number of irreversible vascular diseases such as atherosclerosis, hypertension, stroke, coronary artery disease, congestive heart failure, diabetes, renal failure, pulmonary hypertension, and hyperhomocysteinemia. (Ross, supra; Meredith, supra; J N Cohn, Am J Hypertens 14, 258S (2001); S H Monnink, P L van Haelst, A J van Boven, E S Stroes, R A Tio, T W Plokker, A J Smit, N J Veeger, H J Crijns, W H van Gilst, J Investig Med 50, 19 (2002)). Other conditions that have been associated with endothelial dysfunction include cigarette smoking, hypercholesterolemia, liver cirrhosis, transplantation, acute respiratory distress syndrome (ARDS), erectile dysfunction, postmenopausal state, preeclampsia, and dementia (Paterick, supra; H Vapaatalo, E Mervaala, Med Sci Monit 7, 1075 (2001); J P Granger, B T Alexander, M T Llinas, W A Bennett, R A Khalil, Hypertension 38, 718 (2000); K T McVary, S Carrier, H Wessells, J Urol 166, 1624 (2001)) and chronically non-healing wounds such as chronic diabetic ulcers, venous stasis ulcers, and certain wounds resulting from trauma, surgery, or radiation injury. The early clinical detection of endothelial dysfunction would be useful in predicting the future development of cardiovascular disease, in predicting clinical outcomes for existing medical conditions, and in developing and monitoring treatment protocols for existing medical conditions.

Traditional markers and assays for endothelial dysfunction include direct measurement of NO or its metabolites and functional measurement of vascular NO-dependent responses (Vapaatalo, supra; R Joannides, W E Haefeli, L Linder, V Richard, E H Bakkali, C Thuillez, et al. Circulation 91, 1314 (1995)). Clinical assessment of endothelial dysfunction in humans frequently requires invasive cardiac and peripheral vascular procedures which are time consuming and not without inherent risk. Non-invasive assessment of endothelial dysfunction can be obtained by imaging techniques, blood flow measurements, and measurement of circulating biomarkers such as asymmetric dimethylarginine (ADMA), serum nitrite or nitrate, C-reactive protein, and endothelin (H M Farouque, I T Meredith, Coron Artery Dis 12, 445 (2001)). These determinations offer only an indirect evaluation of endothelial dysfunction and have uncertain clinical value. Currently no method of determining endothelial dysfunction is sufficiently robust for clinical decision making at the individual patient level (Farouque, supra).

NO is a small, hydrophobic, gaseous free radical which is an important physiological mediator for autonomic functions such as vasodilatation, neurotransmission, and intestinal peristalsis. NO provides cellular signaling by activation of its target molecule, guanylate cyclase, which elevates intracellular concentrations of cyclic guanosine monophosphate (cGMP) (J S Beckman, in Nitric Oxide, J. Lancaster, Jr., Ed. (Academic Press, N.Y.), chap. 1). Cellular signaling is performed without mediation of channels or cellular membrane receptors and is dependent upon the concentration of NO in the cellular environment.

NO has a half-life of about five seconds in biological tissues. It is generated by three isoforms of NO synthase (NOS) which metabolize L-arginine and molecular oxygen to citrulline and NO. Two of the three isoforms are constitutive enzyme systems (cNOS) which are described in neuronal cells (nNOS) and in endothelial cells (eNOS) (D Bruch-Gerharz, T Ruzicka, V Kolb-Bachofen. J Invest Dermatol. 110, 1 (1998)). With these isoforms, increased levels of intracellular calcium activate the enzymes via calmodulin. The calcium-dependent cNOS systems produce low (picomolar) concentrations of NO. The third system is the inducible isoform (iNOS) which is calcium independent. The expression of iNOS is induced by tissue-specific stimuli such as inflammatory cytokines or bacterial lipopolysaccharide (LPS). The inducible isoform releases NO in much higher (nanomolar) concentrations than cNOS and has potent cytotoxic effects.

The cNOS enzymes are involved in regulating and maintaining skin homeostasis (S Moncada, A Higgs, N Eng J Med 329, 2002 (1993)). The iNOS enzymes appear to be mainly associated with inflammatory and immune responses that are also implicated in certain skin diseases. In human skin keratinocytes, fibroblasts and endothelial cells possess both the cNOS and iNOS isoforms. The wound macrophage and keratinocyte possess the iNOS isoform. Reduction of iNOS activity has been found to delay wound healing. Enhancement of iNOS activity by adenoviral-mediated topical iNOS gene transfer has been found to reverse delay in closure of excisional wounds in mice with deficient iNOS activity (KYamasaki et al., J. Clin. Invest. 101:967-971 (1998)).

Superoxide is produced in all cells as result of normal oxidative metabolism. Superoxide reacts with NO in a rapid, diffusion limited manner to produce peroxynitrite, which can initiate lipid peroxidation and can react with thiol groups or tyrosine residues in proteins (JS Beckman et al., Am J Physiol 271:C1424 (1996)). Existing levels of superoxide in vivo have been shown to reduce the biological activity of NO (A Mugge et al., Am J Physiol 260:C219 (1991)). Superoxide production is increased in several disease states, e.g., hypercholesterolemia, hypertension, and diabetes mellitus (D. Tomasian et al., Cardiovasc Res 47:426 (2000)). Superoxide can be broken down by the enzyme superoxide dismutase. Increasing the activity of superoxide dismutase or decreasing the production of superoxide has been shown to improve endothelial-dependent vasodilator responses in atherosclerosis and other diseases (S Rajagopalan et al., J Clin Invest 97:1916 (1996); A Mugge et al., Circ Res 69:1293 (1991)).

The bioactivity of NO can be compromised by oxidant stress. Oxidant stress is the excess of oxidants relative to antioxidant defenses (D Tomasian et al., Cardiovasc. Res. 47:426-435 (2000)). Accelerated inactivation of NO by reactive oxygen species such as superoxide anion is thought to be related to endothelial dysfunction in diseases such as diabetes, cigarette smoking, hypercholesterolemia, atherosclerosis, and heart failure (H Cai & DG Harrison, Circ Res 87:840-844 (2000)). NO becomes inactivated by oxidative stress in diabetic human subjects, resulting in microvascular complications (K Maejima et al., J. Diabetes and Its Complications 15:135-143 (2001)). Administration of antioxidants (e.g., raxofelast, a vitamin E-like antioxidant) stimulates wound healing in diabetic mice (M Galeano et al., Surgery 129:467-44 (2001)). Possible sources of reactive oxygen species in diabetes and other conditions include increased lipid peroxidation and factors secreted by inflammatory cells. Oxidation of low density lipoprotein (LDL) by reactive oxygen species leads to atherogenesis, foam cell formation, inflammation, increased expression of cell adhesion molecules, alteration of normal endothelial cell phenotype with loss of NO production, increased production of reactive oxygen species, and further lipid peroxidation (Tomasian, supra). Furthermore, oxidized LDL is toxic to endothelial cells (A Negre-Salvayre et al., Atherosclerosis 99:207 (1992)), reduces eNOS protein levels in endothelial cells (J K Liao et al., J Biol Chem 270:319 (1995)), and recruits inflammatory cells to the vascular wall which in turn produce more reactive oxygen species.

Among the medical conditions which are related to endothelial dysfunction and reduced NO bioactivity, several are characterized by impaired wound healing. Recent research on the role of NO in wound inflammation, tissue repair, and microvascular homeostasis has demonstrated that NO is a primary regulator of wound healing (D Bruch-Gerharz, T Ruzicka, V Kolb-Bachofen. J Invest Dermatol. 110, 1 (1998); MR Schaffer et al., Surgery 121, 513 (1997)). The effectiveness of NO as a regulator of wound healing is determined not only by the biosynthesis of NO but also by its degradation, which is linked to the metabolism of reactive oxygen species.

A systemic deficiency of endothelial-derived NO has been observed in all diabetics (A Veves et al., Diabetes, 47, 457 (1998); M Huszka et al., Thrombosis Res, 86(2), 173 (1997); S B Williams, J A Cusco, M A Roddy, M T Hohnston, M A Creager, J. Am. Col. Cardiol., 27(3), 567 (1996)), suggesting that NO plays a fundamental role in the pathogenesis of chronic, non-healing lower extremity ulcerations (LEU, also known as chronic diabetic ulcers) which are common among diabetics. While the majority of diabetics exhibit “normal” wound healing, those presenting with chronic LEU often demonstrate decreased wound inflammation, recurrent wound infections, decreased cutaneous vascular perfusion, poor wound collagen deposition, and scar maturation. Consequently, there is a need to correlate NO bioactivity with wound healing ability in diabetics. Such a correlation would allow the development of methods to predict the wound healing ability of diabetics based on their production of NO and would provide a useful clinical indicator which could serve as a basis for choosing appropriate therapy.

Another condition characterized by a deficiency of endothelial-derived NO is chronic venous stasis ulceration (VSU). The fundamental derangement in patients with chronic venous insufficiency (CVI) and secondary VSU is sustained venous hypertension derived from valvular incompetence, outflow obstruction, and/or calf muscle dysfunction (E Criado, in Vascular Surgery, ed. R B Rutherford, 4th ed. W. B. Saunders, Philadelphia, pp. 1771-85 (1995)). However the development of the venous ulcer in the CVI patient is related to white cell trapping, in which the sequestered, activated leukocyte becomes a source of proteolytic enzymes and reactive oxygen species that are released within the microcirculation of the affected extremity. This causes endothelial damage, fibrin cuff deposition, and localized tissue ischemia and necrosis (PD Coleridge-Smth, et al., Br Med J 296:1726-7 (1988); PJ Pappas, et al., J Surg Res 59:553-9 (1995)). It has been demonstrated that CVI is associated with increased platelet and leukocyte (i.e., monocyte) activation and aggregation throughout the circulation (BD Peyton et al., J Vasc Surg 27:1109-16 (1998)). However, the presence of factors released from activated platelets and leukocytes is not predictive of patients who develop venous ulcerations (C C Powell et al., J Vasc Surg 30:844-53 (1999)).

Many other conditions which involve poor wound healing ability are thought to involve reduced bioactivity of NO. These include sickle cell disease (J S Mohan et al., Clin Sci (Lond) 92:153 (1997)), radiation therapy and smoking (T K Hunt et al., Adv Skin Wound Care 13:6 (2000)), sepsis (D W Wilmore, World J Surg 24:705 (2000)), aging (H Desai, J Wound Care 6:237 (1997)), and spinal cord injury (S W Becker et al., Spinal cord 39:114 (2001)).

There remains a need in the art for methods of identifying patients having medical conditions associated with endothelial dysfunction and reduced bioactivity of endothelial-derived NO. There also is a need to evaluate the roles of NO biosynthesis and oxidant stress in contributing to NO deficiency in such patients. Further, there is a need to predict and evaluate clinical outcomes and monitor treatment of such patients.

### SUMMARY OF THE INVENTION

The invention provides methods and kits for the diagnosis and treatment of endothelial dysfunction in a subject based on the NO bioactivity index (NOBI) of the subject. The following embodiments are encompassed by the invention.

In one embodiment of the invention a method of determining whether a subject has endothelial dysfunction is provided. The method comprises the step of comparing the nitric oxide bioactivity index in a specimen from the subject with a threshold value. The nitric oxide bioactivity index is defined as the level of a nitric oxide-related product in the specimen divided by the level of an oxidant stress related product in the specimen. If the nitric oxide bioactivity index is above the threshold value the subject does not have endothelial dysfunction, and if the nitric oxide bioactivity index is approximately at or below the threshold value the subject has endothelial dysfunction.

In another embodiment of the invention a method of determining whether a subject has endothelial dysfunction is provided. The method comprises the step of comparing the nitric oxide bioactivity index in a specimen from the subject with a threshold value. The nitric oxide bioactivity index is defined as the level of an oxidant stress related product in the specimen divided by the level of an nitric oxide-related product in the specimen. If the nitric oxide bioactivity index is below the threshold value the subject does not have endothelial dysfunction, and if the nitric oxide bioactivity index is approximately at or above the threshold value the subject has endothelial dysfunction.

In yet another embodiment of the invention a method of treating a subject having a condition related to endothelial dysfunction is provided. The method comprises the steps of determining the nitric oxide bioactivity index using a specimen from the subject according to the method of claim 1 or 2, and treating the subject according to the nitric oxide bioactivity index.

In still another embodiment of the invention a method of monitoring the effectiveness of treatment of a condition related to endothelial dysfunction is provided. The method comprises the steps of (a) treating the patient using a treatment modality selected from the group consisting of (i) administering an antioxidant, (ii) administering a therapeutic agent designed to raise the level of nitric oxide in the patient, (iii) administering or providing instructions for a diet, and (iv) administering a drug that lowers plasma cholesterol; (b) determining the nitric oxide bioactivity index in a specimen from the patient as a measure of the effectiveness of the treatment, wherein the nitric oxide bioactivity index is defined as the level of a nitric oxide-related product in the specimen divided by the level of an oxidant stress related product in the specimen; and (c) comparing the nitric oxide bioactivity index with a threshold that distinguishes whether the patient has endothelial dysfunction, wherein if the nitric oxide bioactivity index is above the threshold value the effectiveness of treatment is sufficient to treat endothelial dysfunction, and if the nitric oxide bioactivity index is approximately at or below the threshold value the effectiveness of the treatment is insufficient to treat endothelial dysfunction.

Another embodiment of the invention provides a kit for determining whether a subject has endothelial dysfunction. The kit comprises either (1) one or more reagents for determining the level of a nitric oxide-related product in a specimen from the subject, (2) one or more reagents for determining the level of an oxidant stress-related product in a specimen from the subject, or (3) both (1) and (2).

### BRIEF DESCRIPTION OF THE DRAWINGS

FIG. 1 presents a schematic representation of the role of nitric oxide (NO) in wound repair regulation. Wound NO mediated “cellular signaling” appears to enhance the inflammatory mediation of repair, wound oxygen availability, and wound matrix remodeling and maturation.

FIG. 2 is a graphical representation of the fasting urine nitrate levels (micromoles per liter) for controls (C), healed diabetics (HD), and unhealed diabetics (UHD) on days 1 and 2 of the study. Results are shown as mean±S.E., with P-values as compared to C (Pc) and HD (PHD) for each day.

FIG. 3 is a graphical representation of the fasting plasma nitrate levels (micromoles per liter) for controls (C), healed diabetics (HD), and unhealed diabetics (UHD) on days 1 and 2 of the study. Results are shown as mean±S.E., with P-values as compared to C for each day except ‡, which compares HD and UHD for Day-2 only.

FIG. 4 depicts the role of NO in promoting wound healing on the one hand and interaction with reactive oxygen species on the other hand. As shown at the bottom of the figure, the maintenance of NO bioactivity, which is determined by the balance between NO synthesis and degradation, controls the ability to heal wounds.

FIG. 5 is a graphic representation of the NO bioactivity index (NOBI). The squares represent control patients or, as indicated, a diabetic patient with normal wound healing. The triangle represents a diabetic patient with a non-healing wound.

### DETAILED DESCRIPTION OF THE INVENTION

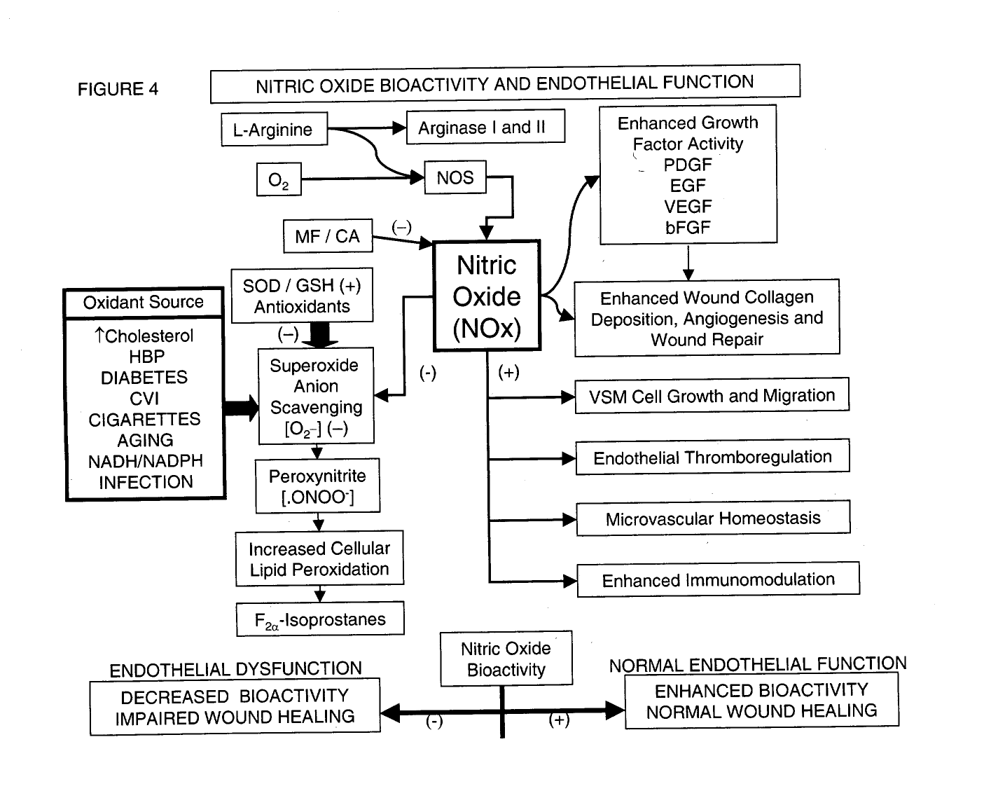

The inventor has discovered certain methods and kits which are designed to detect, treat, and monitor the treatment of patients with endothelial dysfunction. The methods and kits of the invention are based on measurement of the level of an NO-related product and the level of a product related to oxidant stress in a specimen from a patient. Patients represent a continuous spectrum of NO biosynthetic capability. Likewise, patients represent a continuous spectrum of oxidant stress (leading to NO breakdown), which is determined by the inherent metabolism as well as the prevailing physiological and pathological conditions found in each individual patient. The bioactivity (i.e., bioavailability) of NO is determined by the balance between NO synthesis and NO degradation (see FIG. 4) and is dependent on the presence of normal endothelial function. When confronted with conditions of heightened oxidative stress, e.g., high rates of free radical production related to inflammation, diseases such as diabetes, smoking, or lack of antioxidants, a normal patient can compensate for NO breakdown at least in part by increasing endothelial NO synthesis, thereby retaining normal NO bioactivity. Patients who inadequately compensate will have reduced NO bioavailability due to endothelial dysfunction. Thus, patients also represent a spectrum of NO bioactivity. Patients at the lower end of that spectrum are considered to have endothelial dysfunction and may have, or be at risk for developing, any of a variety of medical conditions. The findings of the inventor indicate that below a threshold level of NO bioactivity in a patient, a deficiency of endothelial function is indicated which is diagnostic for the presence of, or for the increased risk of developing, certain medical conditions.

NO bioactivity is directly proportional to NO production and inversely proportional to the production of reactive oxygen species, such as free radicals, which break down NO. Clinical assessment of NO-based endothelial performance (or endothelial dysfunction) according to the invention documents the dynamic equilibrium between endogenous NO production and oxidant stress-related products such as free radicals. Free radical generation, which results in NO scavenging and endothelial cell lipid peroxidation, is the predominant factor responsible for the creation of oxidant stress, lipid peroxidation, and NO degradation.

Superoxide and other oxygen-related free radicals are a source of oxidant stress in the body. Oxidant stress has a negative impact on many physiological processes, such as wound healing. NO reacts avidly with superoxide (.O₂⁻) to yield peroxynitrite (ONO₂⁻). Not only does this reaction reduce the availability of NO in the body, but superoxide, peroxynitrite, and other reactive oxygen species can cause lipid peroxidation which damages endothelial cells and further reduces NO synthesis. The level of an oxidant stress-related product in a specimen is indicative of the level of oxidant stress, e.g., of the rate of production of superoxide, the rate of destruction of NO by reactive oxygen species, the level of lipid peroxidation, and the level of endothelial cell damage in a patient. A number of medical conditions, including diabetes, cigarette smoking, hypercholesterolemia, atherosclerosis, and heart failure (Cai, supra) are characterized by increased oxidant stress and consequent endothelial dysfunction.

The bioactivity of NO can be determined as the NO Bioactivity Index (NOBI). NOBI is the ratio (or inverse thereof) of the level of an NO-related product in a specimen from a patient to the level of an oxidant stress-related product in the same or similar specimen from the same patient. The patient can be either a human or a non-human animal. The NOBI can also be determined for a group of patients, either by averaging the NOBI from each patient, or by averaging the NO-related product level for all patients and dividing it by the average oxidant stress-related product average over all patients. Any desired mathematical representation of a group can be used, such as the mean, median, or other values representative of the group in a mathematically defined way. Individual NOBI values for a group of patients can also be displayed graphically for comparison between patient groups, or for comparison of an individual patient to a group of patients. Statistical analyses such as linear or non-linear regression analyses can also be applied to NOBI data from groups of patients, or can be applied to data for one or more NO-related products and/or one or more oxidant stress-related products. Any desired statistical measure such as standard deviation, standard error, confidence limits, and variance can be employed to compare either groups of patients, individual patients, or groups with individual patients.

NO is normally metabolized to certain stable products such as nitrate and nitrite, which may be assayed in urine, plasma, tissue, wound fluid, or other specimens from a patient. The level of nitrate, nitrite, or other NO-related products in a specimen serves as an indicator of the level of NO synthesis in a patient. Oxidant stress-related products such as isoprostanes are formed by the reaction of superoxide, peroxynitrite, and other reactive oxygen species with membrane lipids (i.e., by lipid peroxidation). Such reactive oxygen species are also responsible for the chemical degradation of NO. Furthermore, lipid peroxidation and other biochemical reactions driven by reactive oxygen species cause damage to the endothelium, which further diminishes the level of NO by reducing NO synthesis. The level of an oxidant stress-related product, such as isoprostane, can be detected by assay of a urine, plasma, tissue, wound fluid, or other specimen from a patient. The NOBI takes into account the balance between NO synthesis and NO destruction resulting from oxidant stress. The NOBI is therefore an indicator of the bioavailability and effectiveness of NO in promoting vascular health and likewise an indicator of endothelial performance. Generally, the higher the NOBI, the greater the bioactivity of NO and the lower the probability that endothelial dysfunction exists in a patient. The lower the NOBI, the lower the bioactivity of NO and the greater the endothelial dysfunction in a patient.

NO-Related Products

A variety of molecular species related to NO synthesis or breakdown (“NO-related products”) can be quantified in blood, urine, tissue, or other samples from a patient. The major metabolic pathway for NO is to nitrate and nitrite, which are stable metabolites within tissue, plasma, and urine (S Moncada, A Higgs, N Eng J Med 329, 2002 (1993)). Tracer studies in humans have demonstrated that perhaps 50% of the total body nitrate/nitrite originates from the substrate for NO synthesis, L-arginine (P M Rhodes, A M Leone, P L Francis, A D Struthers, S Moncada, Biomed Biophys Res. Commun. 209, 590 (1995); L. Castillo et al., Proc Natl Acad Sci USA 90, 193 (1993)). Although nitrate and nitrite are not measures of biologically active NO, plasma and urine samples obtained from subjects after a suitable period of fasting, and optionally after administration of a controlled diet (low nitrate/low arginine), allow the use of nitrate and nitrite as an index of NO activity (C Baylis, P Vallance, Curr Opin Nephrol Hypertens 7, 59 (1998)).

Plasma levels of L-citrulline, which is a product of the reaction that produces NO, or cGMP, which is produced as a result of NO activation of guanylate cyclase, can be determined as a reflection of systemic NO synthesis in a patient. (F L Kiechle and T Malinski, Ann. Clin. Lab. Sci. 26, 501 (1996)). Similarly, L-dimethylarginine, another product of NOS, can be detected by HPLC of human serum and used as a highly specific index of systemic NOS activity. (J Meyer et al., Anal. Biochem. 247, 11 (1997)). NO can also break down by reacting with superoxide anion in human plasma to produce peroxynitrite, which in turn can produce a variety of radicals such as ascorbyl radical and albumin-thinyl radical that can be detected using electron paramagnetic resonance (EPR) spectroscopy. (J Vasquez-Vivar et al., Biochem. J. 314, 869 (1996)). Another product of peroxynitrite is 3-nitrotyrosine, which can be detected in human plasma or other fluids by gas chromatography in tandem with mass spectrometry (E Schwedhelm et al., Anal. Biochem. 276, 195 (1999)), reversed-phase HPLC (H Ohshima et al., Nitric Oxide 3, 132 (1999)), or an ELISA method using anti-nitrotyrosine antibodies (J C ter Steege et al., Free Radic. Biol. Med. 25, 953 (1998)). Unlike nitrate or nitrite, most of these products are not subject to interference by dietary intake. Furthermore, the in situ detection of NO itself is possible with the aid of biosensors that quantify NO levels and changes in NO levels in response to stimuli. For example, the heme domain of soluble guanylate cyclase, a natural receptor for NO, can be labeled with a fluorescent reporter dye, and changes in fluorescence intensity can be determined through an optical fiber and calibrated to reveal NO levels at any desired location in the body, for example at or near a wound site (S L Barker et al., Anal. Chem. 71, 2071 (1999)). Given the rapid decomposition of NO in biological fluids, direct detection of NO should be performed in situ rather than some time following collection of a specimen.

The level of nitrate or nitrite in the specimen can be quantified by any method known in the art which provides adequate sensitivity and reproducibility. For example, the Griess reaction is a spectrophotometric assay for nitrate which can provide sensitive determination of nitrate and nitrite in biological fluid samples (M Marzinzig et al., Nitric Oxide 1, 177 (1997)). If the Griess reaction or another nitrite assay is performed both with and without reduction of nitrate to nitrite, then nitrate values can be obtained as the difference between the nitrite values obtained for the reduced sample and the non-reduced sample. The Griess assay can be made more sensitive if a fluorescent product is obtained, e.g., by reacting nitrite with 2,3-diaminonaphalene (T P Misko et al., Anal. Biochem. 214, 11 (1993)). Highly sensitive assays are also available which first reduce nitrite and nitrate (RS Braman and S A Hendrix, Anal. Chem. 61, 2715 (1989)) or any NO-related compound (M Sonoda et al., Anal. Biochem. 247, 417 (1997)) to NO for detection with specific chemiluminesence reagents. A variety of protocols have also been described for detecting and quantifying nitrite and nitrate levels in biological fluids by ion chromatography (e.g., S A Everett et al., J. Chromatogr. 706, 437 (1995); J M Monaghan et al., J. Chromatogr. 770, 143 (1997)), high-performance liquid chromatography (e.g., M Kelm et al., Cardiovasc. Res. 41, 765 (1999)), and capillary electrophoresis (M A Friedberg et al., J. Chromatogr. 781, 491 (1997)).

The “level” of NO-related product or oxidant stress-related product usually refers to the concentration (in moles per liter, micromoles per liter, or other suitable units) of the respective product in the specimen, or in the fluid portion of the specimen. However, other units of measure can also be used to express the level of the products. For example, an absolute amount (in micrograms, milligrams, nanomoles, micromoles, moles, or other suitable units) can be used, particularly if the amount refers back to a constant amount, mass, or volume of patient specimen (e.g., grams, kilograms, milliliters, liters, or other suitable units). A number of commercially available kits can be used. One such kit is described in Example 2.

Oxidant Stress-Related Products

A variety of molecular species can be determined as “oxidant stress-related products,” including, but not limited to, isoprostanes, malondialdehyde, conjugated dienes, thiobarbituric acid reactive substances, 4-hydroxynonenal, oxidized low density lipoprotein, serum lipid peroxide, and advanced glycation end products (AGEs). Isoprostanes (e.g., 8-epi-prostaglandin F₂ₐₗₚₕₐ) are preferred oxidant stress-related products. Isoprostanes are chemically stable products that result from the non-enzymatic reaction of arachidonic acid with oxygen radicals. The F₂ isoprostanes are a sensitive, direct marker of in vivo cellular oxidative damage caused by free radicals (i.e., a marker for lipid peroxidation). F₂ isoprostanes are also a marker for reactive oxygen species, which promote the degradation of NO and thereby reduce its bioactivity. F₂ isoprostanes are stable eicosanoids which are generated in conditions of increased oxidative stress by the enzyme-independent free radical oxidation of arachidonic acid in membrane phospholipids and lipoproteins. The F₂ isoprostanes may also independently participate in oxidative injury. They are characterized by biological activities mediated by the endothelium which antagonize NO. Such functions include platelet activation, increased platelet adhesiveness, and platelet aggregation, as well as constriction of the renal and pulmonary vasculature. The F₂ isoprostanes are generally regarded as an accurate means of clinically quantifying lipid peroxidation and oxidant stress.

Isoprostane levels in plasma and in some cases in urine are increased in pathogenic conditions caused by oxidant stress and are considered a reliable marker for oxidant stress (C Souvignet et al., Fundam Clin Pharmacol 14:1 (2000); T A Mori et al., Anal Biochem 268:117 (1999)). Antioxidants such as alpha tocopherol have been shown to reduce such isoprostane levels in biological fluids (C Souvignet et al., Fundam Clin Pharmacol 14:1 (2000)). Urinary isoprostane levels are significantly higher in smokers than in non-smokers, showing that isoprostane levels in specimens from a subject correlate with oxidant stress (T Obata et al., J Chromatogr B Biomed Sci Appl 746:11 (2000)). Women with preeclamptic pregnancy show elevated isoprostane levels in plasma but not in urine (E T McKinney et al., Am J Obstet Gynecol 183:874 (2000)). Isoprostane levels in plasma of diabetic men was about five-fold higher than in controls, and the isoprostane levels in the diabetics fell by 50% in response to treatment with raxofelast (600 mg twice daily for seven days; P J Chowienczyk et al., Diabetologia 43:974 (2000)). Raxofelast is a synthetic, water soluble antioxidant which is an analogue of alpha tocopherol. Raxofelast, which is 2-(2,3-dihydro-5-acetoxy-4,6,7-trimethylbenzofuranyl) acetic acid (IRFI 016), is converted in the body to an active metabolite, 2-(2,3-dihydro-5-hydroxy-4,6,7-trimethylbenzofuranyl) acetic acid (IRFI 005).

Methods of detecting oxidant stress-related products are likewise known in the art. For example, 8-epi-PGF2alpha, one of the most abundant isoprostanes, can be quantified in plasma and urine using silica and reverse phase HPLC followed by gas chromatography-mass spectrometry (TA Mori et al., Anal Biochem 268:117 (1999)). Alternatively, an enzyme immunoassay kit for determination of 8-isoprostane is commercially available (Cayman Chemical cat. no. 516351). Plasma specimens from healthy human subjects typically contain about 40-100 pg/ml of 8-isoprostane, while urine specimens from healthy humans contain about 10-50 ng of 8-isoprostane per mmol of creatinine (Wang et al., J Pharmacol Exp Ther 275:94 (1995); MP Reilly et al., Fibrinolysis & Proteolysis 11:81 (1997)). Several assays exist for malondialdehyde (MDA) in plasma, urine, and other specimens. Such assays include specific reagents for UV detection by HPLC (J P Steghens et al., Free Radic Biol Med 31:242 (2001) and J Pilz Chromatogr B Biomed Sci appl 742:315 (2000)) and capillary electrophoresis (K N Korizis et al., Biomed Chromatogr 15:287 (2001)). A variety of lipid peroxidation products including MDA can be quantified using the thiobarbituric acid reaction (K Fukanaga et al., Biomed Chromatogr 12:300 (1998)). Another by product of lipid peroxidation which can be detected in specimens is 4-hydroxy-2-nonenal (HNE). HNE can be detected using antibodies (N Tanaka et al., Arch Dermatol Res 293:363 (2001)) or derivitization with a fluorescent reagent followed by micellar electrokinetic chromatographic separation and laser-induced fluorescence detection (K Claeson et a., J Chromatogr B Biomed Sci Appl 763:133 (2001)). Oxidized LDL can be quantified by immunohistochemical techniques (Q Javed et aL, Exp Mol Pathol 65:121 (1999)) and by reaction with thiobarbituric acid (M Tanaka et al., Biol Pharm Bull 16:538 (1993)). Advanced glycation end products (AGEs), also known as advanced Maillard products, are irreversibly glycated proteins which catalyze the formation of free radicals. Their presence is indicative of oxidant stress in old age, atherosclerosis, diabetes, and other conditions related to endothelial dysfunction. AGEs can be detected as outlined by MB Yim et al., Ann N Y Acad Sci 928:48 (2001) and references described therein.

Diagnosis of Endothelial Dysfunction by NOBI

The NOBI can be determined using any selected NO-related product together with any selected oxidant stress-related product. There is no limitation with respect to which NO-related product can be combined with which oxidant stress-related product. However, certain products may be chosen based on availability or reliability of detection methods, based on the presence of accurately quantifiable levels of a product in a particular type of patient specimen, based on experience with given products or detection methods, or based on suitability to differentiate between particular types of patients or medical conditions. In a preferred embodiment, for example, endothelial NO synthesis and metabolism are quantified as plasma nitrate concentration, and oxidant stress is quantified as plasma concentration of isoprostanes.

The determination of NOBI can be applied to patients suffering from any type of medical condition related to endothelial dysfunction. A patient or subject according to the invention can be any human or animal having or suspected of having a condition related to endothelial dysfunction. By “condition related to endothelial dysfunction” is meant any condition which is caused by endothelial dysfunction in whole or in part, or any condition which itself causes endothelial dysfunction. Such conditions include, but are not limited to, diabetes mellitus (type I or type II), preclinical diabetes, hypertension, atherosclerosis, atherosclerotic peripheral vascular disease, chronic diabetic ulcer, venous stasis ulcer, decubitus ulcer, steroid-dependent ulcer, chronic venous insufficiency, sickle cell disease, trauma, chronic non-healing surgical wound, chronic non-healing burn injury, chronic osteomyelitis, erectile dysfunction, postmenopausal state, preeclampsia, cigarette smoking, acute respiratory distress syndrome (ARDS), radiation injury, spinal cord injury, malnutrition, sepsis, chronic soft tissue infection, vitamin deficiency, osteoporosis, post-operative surgical wound, old age (age greater than 75 years), cigarette smoking, and any condition that elevates oxidant stress or causes lipid peroxidation. Endothelial dysfunction can be caused, for example, by injury or destruction of endothelial tissue by any means which degrades or eliminates the function of endothelial cells, resulting in diminution of their ability to synthesize NO, reduction in the rate of NO synthesis, impaired regulation of NO synthesis, or reduced expression of one or more NO synthetic enzymes.

The balance between endothelial NO synthesis and NO degradation caused by reactive oxygen species, i.e., endothelial redox equilibrium, can be defined by the NOBI determined in a control setting with healthy subjects. Healthy control subjects are those which show no symptoms of a medical condition caused by endothelial dysfunction. Healthy control subjects preferably have normal NO synthetic capability and are free of abnormal oxidant stress and lipid peroxidation. In healthy subjects, NOBI is an expression of the normal relationship (i.e., normal redox equilibrium) between NO production and the level of reactive oxygen species (e.g., free radicals). Endothelial NO synthetic ability can be estimated as the level of an NO-related product in a sample from a subject. The activity of reactive oxygen species and the rate of NO degradation caused by reactive oxygen species can be estimated as the level of an oxidant stress-related product in a sample from a subject. The NOBI is expressed as the ratio of these two factors. For example, NOBI is the ratio of an NO-related product to an oxidant stress-related product. NOBI can also be expressed as the ratio of an oxidant stress-related product to an NO-related product. NOBI can also be determined as the slope of a plot of one factor vs. the other factor. For example, NOBI can be expressed as the slope of a plot of an NO-related product on the vertical axis and an oxidant stress-related product on the horizontal axis. Alternatively, NOBI can be expressed as the slope of a plot of an oxidant stress-related product on the vertical axis and an NO-related product on the horizontal axis. Subjects with endothelial dysfunction can be identified by the deviation of their NOBI from that representing normal endothelial redox equilibrium, i.e., deviation from the NOBI of healthy control subjects. The presence of either deficient NO production or increased lipid peroxidation, either of which would depress NO bioactivity, can be observed as an alteration of the NOBI from that observed during normal endothelial redox equilibrium.

According to the invention, patients with endothelial dysfunction can be distinguished from patients with normal endothelial function by comparing the NOBI of a test subject to the NOBI of healthy control subjects. Endothelial dysfunction is diagnosed in a test subject if the NOBI of the test subject differs appropriately from the NOBI of healthy control subjects. Thus, if NOBI is expressed as the level of an NO-related product divided by the level of an oxidant stress-related product, then a test subject whose NOBI is numerically smaller than that of healthy control subjects is diagnosed as having endothelial dysfunction. Conversely, if NOBI is expressed as the level of an oxidant stress-related product divided by the level of an NO-related product, then a test subject whose NOBI is numerically larger than that of healthy control subjects is diagnosed as having endothelial dysfunction. In order to facilitate the diagnosis of endothelial dysfunction in test subjects, a threshold value of NOBI can be identified which separates healthy control subjects from subjects with endothelial dysfunction.

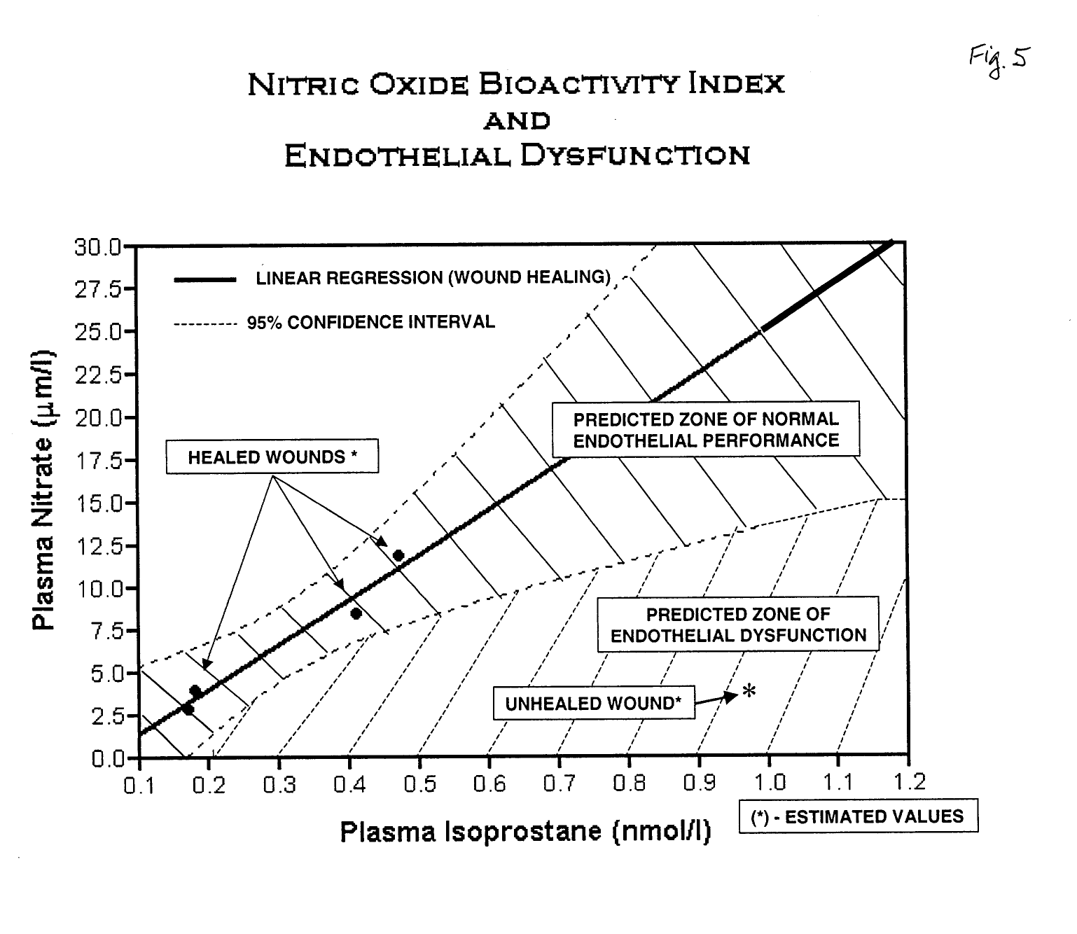

The threshold value of normal NOBI can be determined by comparing a group of subjects with normal endothelial function to another group of subjects with endothelial dysfunction. Preferably, all of the subjects in the group with endothelial dysfunction share a common medical condition related to endothelial dysfunction. An example of an experiment which can be used to identify an NOBI threshold is presented in Example 3. By determining the plasma nitrate levels and isoprostane levels of a group of healthy control subjects or wound healing diabetics with a group of non-wound healing diabetics, preferably following the administration of a low nitrate diet and after a fasting period, the NOBI of the two groups can be compared (see FIG. 5). The threshold value can be selected from the data obtained.

Assuming NOBI is calculated as the level of an NO-related product divided by the level of an oxidant stress-related product, then the threshold chosen will define the lower limit of the normal range of NOBI values. The threshold can be chosen as a value higher than the mean of the NOBI of the group with endothelial dysfunction. The threshold value should be chosen such that the NOBI of at least 70%, 80%, 90%, 95%, 98%, or 99% or more of the patients with endothelial dysfunction would fall at or below the threshold. Alternatively, the threshold can be selected as a value below the mean NOBI for the healthy control subjects. For example, the threshold can be chosen as the mean of the control group minus an appropriate statistical measure, such as the standard error of the mean for the control group, a desired multiple (e.g., one, two, three, or more) of the standard deviation for the control group data, or a specified confidence interval (e.g., 80%, 85%, 90%, 95%, 98%, or 99% confidence interval) for the control group data. For human patients, the threshold value for normal NOBI is between 10 and 40 micromoles of plasma nitrate per nanomole of plasma isoprostane. Preferably, the threshold value for normal human NOBI is between 20 and 30 or between 23 and 27 micromoles plasma nitrate per nanomole plasma isoprostane. More preferably, the threshold value for normal human NOBI is about 20, 23, 25, 28, 30, 32, 35, 37, or 40 micromoles plasma nitrate per nanomole plasma isoprostane. When selecting a threshold value of NOBI for use with a given type of specimen, for example human urine or plasma, it should be noted that the use of different NO-related products, different oxidant stress-related products, different detection assays, different units, or different methods of standardization could alter the specified numerical ranges. Furthermore, if NOBI is calculated as the level of an oxidant stress-related product divided by the level of an NO-related product, i.e., the reciprocal of the calculation described above, then the threshold chosen will define the upper (not lower) limit of the normal range of NOBI values, and all numerical NOBI values stated earlier in this paragraph should be substituted with their reciprocal values.

In some circumstances, two-dimensional analysis of NOBI allows the clinician to implement therapies to correct individual underlying factors that contribute to endothelial dysfunction. For example, a patient with endothelial dysfunction might benefit more from antioxidant therapy if the patient reveals a high oxidant stress level than if the patient has a low rate of NO synthesis. Conversely, a patient with low NO synthetic rate might respond better to L-arginine therapy than a patient with high oxidant stress. Certain patients can better be distinguished when the NOBI component data, i.e., the level of an NO-related product and the level of an oxidant stress-related product, are plotted on separate axes, i.e., in two dimensions, and compared to the normal range of NOBI values on the same graph. See, for example, FIG. 5. Normal NOBI values ordinarily will be distributed as a band (ideally a line) whose slope corresponds to the average normal NOBI. Plotting the NOBI for an individual patient or for a group of patients who share a particular medical condition related to endothelial dysfunction can shed insight into the nature of the defect responsible for endothelial dysfunction. In particular, two dimensional analysis can reveal whether the defect is dominated by either a deficit in NO synthesis or an excess of oxidant stress. If a patient or group average NOBI is displaced from the normal NOBI curve more along the axis representing the NO-related product, then the predominant defect is more likely to be one of inadequate NO synthesis. If, on the other hand, the patient or group average NOBI is displaced from the normal NOBI curve more along the axis representing an oxidant stress-related product, then the predominant defect is more likely to be one of excess reactive oxygen species, excess free radicals, or insufficient antioxidant defenses.

NOBI Predicts Failure to Compensate for Oxidant Stress

NOBI is a unique parameter which serves as a reporter of endothelial dysfunction. NOBI enables novel methods for the diagnosis and treatment of medical conditions associated with endothelial dysfunction. In a patient with normal endothelial function, the degradation of NO during periods of heightened oxidant stress can be compensated, at least in part, by increased NO production. Such compensation is observed in diabetic patients whose wounds heal normally. On the other hand, diabetic patients with impaired wound healing have a significantly decreased NOBI compared to control patients or healed diabetics. See Example 3. Note that NOBI is numerically decreased in patients with endothelial dysfunction if NOBI is expressed as the ratio of an NO-related product to an oxidant stress related product; NOBI would be numerically increased in such patients if NOBI is expressed as the reciprocal of that ratio. Compensation for oxidant stress by increased NO production acts to preserve the endothelial environment by maintaining NO bioactivity within a physiologically acceptable range during increased oxidant stress. This protective feature of the vascular environment maintains, for example, optimal NO-mediated vasomotor tone and endothelial thromboregulation while suppressing platelet activation in an environment of increasing plasma free radical activity. Subjects with endothelial dysfunction have an impaired ability to compensate for oxidant stress through enhancement of NO production. Such an impairment can be manifested as a poor clinical outcome in a variety of medical conditions, e.g., poor wound-healing ability, chronic inflammation, accelerated development of atherosclerosis, hypertension, or other cardiovascular ailments.

Acute hyperglycemia has been demonstrated to significantly increase oxidant stress and lipid peroxidation, determined as plasma 8-epi-PGF₂ₐₗₚₕₐ isoprostane, in persons with type 2 diabetes (M J Sampson, et al., Diabetes Care 25:537 (2002)). Diabetic patients with normal endothelial function can compensate for such increased oxidant stress by increasing the production of nitric oxide. A recent study found elevated plasma nitrate levels which correlated with increased serum advanced glyation end products (AGEs, a marker of oxidant stress) in diabetic patients compared to non-diabetic patients (K Maejima et al., J. Diabetes Complications 15:135 (2001)).

Diabetic patients with endothelial dysfunction cannot fully compensate for such oxidant stress, however. In the clinical diabetic studies described in Examples 1 and 2 below, the state of cellular activation and increased production of reactive oxygen species was documented as a significantly increased level of fasting plasma nitrate in the wound healing diabetic patients that was not observed in the non-wound healing diabetic patients. From these observations, it is evident that the wound healing diabetic is capable of initiating an effective compensatory increased endothelial production of NO. This promotes the non-toxic metabolism of increased reactive oxygen species (R M Clancy, et al., J Clin Invest 90:1116-21 (1992); D Wink, et al., Proc Natl Acad Sci USA 90:9813-17 (1993); O'Byrne et al., Diabetes 49:857 (2000)) and maintains the constitutive integrity of NO-mediated wound repair mechanisms.

Thus, NOBI can be used to diagnose the inability of a given patient or set of patients to compensate for the increased oxidant stress encountered in certain medical conditions. If a patient's NOBI is reduced compared to healthy control subjects, i.e., if the ratio of NO-related product to oxidant stress-related product is lower than normal, then the patient lacks the normal compensatory increase of NO synthesis during periods of increased oxidant stress. Furthermore, for some medical conditions individual patients can be challenged by placing them in a state of oxidant stress for a test period (i.e., subjected to an oxidant stress challenge), during which their NO synthesis and metabolism can be analyzed by periodic testing of specimens for NO-related products such as nitrate. For example, high glucose levels in diabetic patients increase the production of reactive oxygen species. One test for diagnosing a patient's compensatory ability to increase NO synthesis in response to oxidant stress is to raise the patient's blood glucose level (e.g., through administration of an oral 75 g glucose tolerance test, see Sampson, supra) and observe either plasma or urinary nitrate, or another NO-related product, as a function of time. Normal patients and patients without significant endothelial dysfunction will respond to high blood glucose levels with increased NO synthesis and consequently increased plasma and urinary nitrate over time. Patients with endothelial dysfunction, who have below normal compensation for oxidant stress, will produce a weaker than normal rise in plasma and urinary nitrate levels. In another embodiment, both an NO-related product and an oxidant stress-related product are measured in a sample from a patient following a glucose tolerance test. Patients with normal endothelial function will maintain the NOBI in a normal range, whereas patients with endothelial dysfunction will demonstrate a failure to completely compensate for tha added oxidant stress by revealing an NOBI outside the normal range. Such patients are identified as being at risk for developing a medical condition related to endothelial dysfunction, even if they do not reveal evidence of such a condition at the time of the oxidant stress challenge.

Application of NOBI to Wound Repair

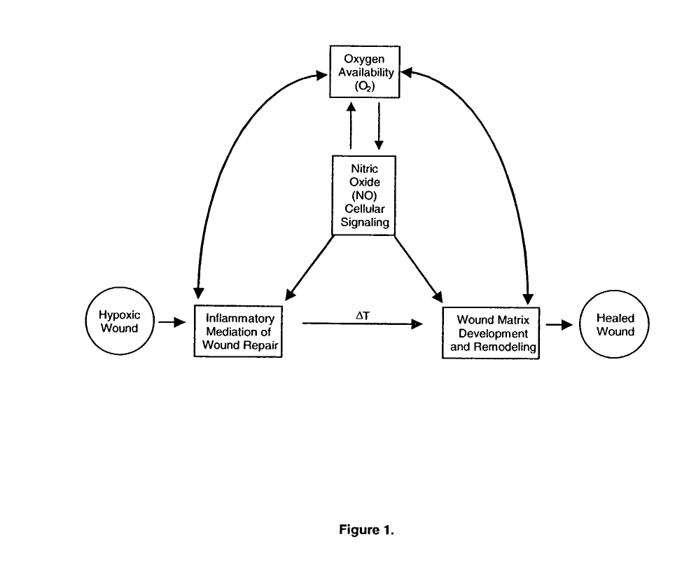

In patients suffering from endothelial dysfunction, normal wound repair can be significantly compromised. In general, during the wound healing process, NO provides enhancement of tissue oxygen availability, the inflammatory mediation of repair mechanisms, and wound matrix development and remodeling (FIG. 1). In wound healing studies NO synthesis has been shown to occur for prolonged periods (10-14 days) after wounding, and macrophages appear to be the major cellular source (M R Schaffer, U Tantry, R A vanWesep, A Barbul. J Surg Res, 71, 25 (1997)). As a mediator of tissue repair, NO has been demonstrated to promote angiogenesis (A Papapetropoulos, G Garcia-Cardena, J A A Madri, W C Sissa. J Clin Invest, 100(12), 3131 (1997)) and cellular migration (E Noiri et al., Am. J. Physiol. 279:C794 (1996)), increase wound collagen deposition and collagen cross-linking (M R Schaffer, U Tantry, S S Gross, H L Wasserburg, A Barbul. J Surg Res, 63, 237 (1996)), regulate microvascular homeostasis (vasodilatation) (D Bruch-Gerharz, T Ruzicka, V Kolb-Bachofen. J Invest Dermatol. 110, 1 (1998)), inhibit platelet aggregation (J S Beckman, in Nitric Oxide, J. Lancaster, Jr., Ed. (Academic Press, N.Y.), chap. 1), inhibit the formation of endothelial-leucocyte adhesions (A M Lefer, D J Lefer, Cardiovascular Res. 32, 743 (1996)), modulate endothelial proliferation and apoptosis (Y H Shen, X L Wang, D E Wilcken, FEBS Lett, 433(1-2), 125 (1998)), increase the viability of random cutaneous flaps (S C Um et al., Plast Reconstr Surg. 101 785 (1998); G F Pierce et al., Proc Natl Acad Sci USA. 86, 2229 (1989)), and enhance cellular immunomodulation and bacterial cytotoxicity (J S Beckman, in Nitric Oxide, J. Lancaster, Jr., Ed. (Academic Press, N.Y.), chap. 1).

The bioactivity of NO is a critical factor for wound healing. In conditions characterized by chronically non-healing wounds in some patients, the cutaneous microcirculation is heavily populated with activated leucocytes which aggregate with activated platelets or the endothelial surface, releasing reactive oxygen species and proteolytic enzymes capable of causing cellular injury and lipid peroxidation. The injurious effects resulting from leukocyte and platelet activation can be ameliorated by compensatory NO-mediated mechanisms responsible for the promotion of endothelial integrity and microvascular homeostasis (Peyton, et al., supra; Powell et al., supra; S Schroder, et al., Am J Pathol 139:81-100 (1991); Z Pecsvarady Z et al., Diabetes Care 17:57-63 (1994); M Huszka et al., Thrombosis Res 86:173-80 (1997)). However, since NO is destroyed by reactive oxygen species, the high level of leukocyte and platelet activation which exists in wound tissues tends to defeat the compensatory NO-mediated mechanisms, creating in effect an NO-limited healing process. Patients who are wound healers are able to compensate for the NO destroyed by activated leukocytes and platelets, whereas patients who are non-wound healers are not able to adequately compensate.

Patients suffering from endothelial dysfunction show distinct similarities in the inflammatory cellular pathology responsible for impaired wound healing. Thus, impaired wound healing in such patients is related to a failure of compensatory repair mechanisms involving endogenous NO production. Wound-healing and non-wound healing patients with any other condition characterized by poor cutaneous wound healing in some patients related to a high level of activation by inflammatory cells can be identified by the analysis of NO-related products described here. For example, two further conditions characterized by chronically impaired cutaneous wound healing in some patients are pressure sores (also referred to as pressure ulcers, decubitus ulcers, or bedsores) and non-healing cutaneous surgical wounds. A non-healing cutaneous surgical wound is any wound identified as such by a surgeon. Alternatively, a non-healing cutaneous surgical wound is a wound formed by incision through the skin which at its edges lacks adhesion and scar tissue formation at about 7 days or more after surgery. A post-operative surgical wound is a wound formed as a result of surgery. Surgery includes any surgical procedure wherein a surgeon creates a surgical wound. A surgical wound is formed by an incision through the skin. Patients with impaired microcirculation, e.g., patients with endothelial damage resulting in reduced constitutive production of NO, are predisposed to develop pressure sores during periods of recurrent illness. M R Bliss, J Tissue Viability 8, 4-13 (1998). Non-healing surgical wounds can have a similar underlying cause, namely endothelial damage leading to reduced constitutive NO synthesis, or they can be related to other types of tissue damage, such as that caused by radiation therapy. Radiation therapy has been linked to endothelial dysfunction and impaired NO synthesis, resulting in vascular stenosis and poor surgical wound healing. T Sugihara et al., Circulation 100, 635-41 (1999).

The invention provides a method of determining whether a subject with a condition characterized by chronically impaired cutaneous wound healing in some patients is a wound healer or a non-wound healer. A “wound healer” refers to a subject whose wound healing capability is approximately the same as that of a normal, healthy subject. A “non-wound healer” refers to a subject whose wound healing capability is reduced from that of a normal, healthy subject and who consequently is at risk for chronic wounds or ulcerations. For example, in one clinical study, non-wound healing diabetics were considered to be those patients with a history of one or more diabetic foot ulcers with incomplete healing after 20 weeks of Regranex® treatment (see Example 1).

Use of NOBI in Diagnosis and Treatment

NOBI can be employed in a variety of situations to diagnose the existence and extent of endothelial dysfunction in a subject. It can also be used to evaluate the likelihood that a subject will develop in the future a medical condition related to endothelial dysfunction. Further, NOBI can be used to decide on and monitor a course of treatment for a patient with a medical condition related to endothelial dysfunction.

One method of determining whether or not a subject has endothelial dysfunction comprises the step of comparing the subject's NOBI to a threshold value that discriminates between wound healers and non-wound healers. The NOBI is the quotient obtained by dividing the level of NO-related product in the specimen by the level of oxidant stress-related product in the specimen, or alternately NOBI can be expressed as the reciprocal of that quotient. In some embodiments, the method further comprises the step of determining the level of an NO-related product in a specimen from the subject. In other embodiments, the method further comprises the step of determining the level of an oxidant stress-related product in the specimen. In yet other embodiments, the method further comprises the step of dividing the level an NO-related product by the level of an oxidant stress-related product, or dividing the level of an oxidant stress-related product by the level of an NO-related product to determine the NOBI of the subject. In still other embodiments, the method further comprises collecting a specimen from the subject.

The specimen can be any sample of fluid or tissue obtained from the subject in sufficient amount as to allow the determination of the level of nitrate, nitrite, or other NO-related product. For example, the specimen can be a sample of urine, blood (including plasma), wound fluid, or tissue. The specimen can be processed prior to determination of nitrate or nitrite as required by the quantification method, or in order to improve the results, or for the convenience of the investigator. For example, processing can involve centrifuging, filtering, or homogenizing the sample. If the sample is whole blood, the blood can be centrifuged to remove cells and the nitrate or nitrite assay performed on the plasma or serum fraction. If the sample is tissue, the tissue can be dispersed or homogenized by any method known in the art prior to determination of nitrate or nitrite. It may be preferable to remove cells and other debris by centrifugation or another method and to determine the nitrate or nitrite level using only the fluid portion of the sample, or the extracellular fluid fraction of the sample. The sample can also be preserved for later determination, for example by freezing of urine or plasma samples. When appropriate, additives may be introduced into the specimen to preserve or improve its characteristics for use in the nitrate or nitrite assay.

The specimen is preferably obtained from the subject after a period of fasting, in order to allow the level of nitrate, nitrite, or other NO-related products to achieve a stable baseline level. The period of fasting reduces interference from dietary and metabolic sources of nitrate or nitrite that are not related to NO breakdown. During the period of fasting, the subject's consumption of all solid and liquid food is reduced from his average consumption by at least 50%, 60%, 70%, 80%, 90%, or 100%. Preferably the subject's consumption of all solid and liquid food is reduced by at least 90%. More preferably the subject's consumption of all solid and liquid food is reduced by 100%. Most preferably, the subject does not consume any solid or liquid food during the fasting period. The subject's consumption of water generally is not restricted during the fasting period; however in some embodiments, the subject also consumes no water during the fasting period. The fasting period should be of sufficient duration as to allow a stable baseline to be achieved in whatever parameter is to be measured. A stable baseline is the condition in which the parameter measured, e.g., urinary nitrate, is generally reproducible and not subject to large fluctuations between repeated measurements or undue interference from dietary, metabolic, or other sources that are not related to NO metabolism. Preferably the period of fasting is at least 3, 4, 5, 6, 7, 8, 9, 10, 12, 16, 20, or 24 hours. More preferably the period of fasting is from 4 to 12 hours, or from 6 to 10 hours, or from 8 to 10 hours, or from 10 to 12 hours.

It is understood that any requirement for fasting will depend upon which NO-related product is being quantified, because some such products are hardly affected by diet; others may require only a brief fast. For example, plasma L-dimethylarginine (J Meyer et al., Anal. Biochem. 247, 11 (1997)) is unaffected by diet, whereas urinary nitrate/creatinine ratios are unaffected by diet if an overnight fast is performed prior to collecting the specimen (PS Grabowski et al., Arthritis Rheum. 39, 643 (1996)).

In some embodiments, the period of fasting is immediately preceded by a period during which the subject is administered a diet that is sufficiently low in sources of dietary nitrate or nitrite to achieve a stable baseline value of whichever NO metabolite will be determined in the specimen. For example, the diet can be one from which all vegetables and nitrate- or nitrite-preserved foods have been eliminated. The diet can also have a reduced level of L-arginine compared to the subject's normal diet. For example, one diet provides a level of nitrate of less than 900 mg/kg body weight/day, and a level of nitrite of less than 9 mg/kg body weight/day.

Another embodiment of the invention is a method for treating a subject with a condition related to endothelial dysfunction. In order to practice this embodiment, the subject's NOBI is first determined. Then, a therapy is developed using the NOBI information. Since subjects suffering from a medical condition related to endothelial dysfunction, as identified by the invention, suffer from reduced NO bioactivity, they can be treated by any therapy which is designed to increase NO bioactivity, i.e., any therapy designed to increase NO production or reduce oxidant stress. Such therapies include, but are not limited to administering L-arginine to the subject, administering an NO-releasing agent to the subject, administering an antioxidant to the subject, administering to the subject a gene transfer vector comprising a polynucleotide encoding an iNOS enzyme, performing hyperbaric oxygen therapy on the subject, administering to the subject a drug that lowers plasma cholesterol or triglycerides, and administering a diet to the subject or instructing the subject to adhere to a diet.

Administration of L-arginine can be through increasing its presence in the diet, oral administration of a dietary supplement comprising L-arginine in any pharmaceutically acceptable form, or parenteral or intravenous injection of a pharmaceutically acceptable preparation comprising L-arginine. The dosage can be selected from any protocol known in the art which is designed to increase NO production in the patient. A further therapy which increases NO production is hyperbaric oxygen therapy. Yet another therapy which increases NO production is the administration of a gene transfer vector containing a polynucleotide encoding a functional iNOS enzyme. For example, an adenoviral vector can be prepared which delivers the human iNOS gene, and the vector can be administered topically at the site of a wound. See, for example, K Yamasaki et al., J. Clin. Invest. 101:967-971 (1998). A variety of suitable techniques for transfer of an iNOS gene are well known in the art. Yet another possible therapy involves the application of an NO releasing agent. Such agents are known in the art and can be applied topically or by injection at the site of the wound. For example, a linear phenylethyleneimine-NO adduct can be employed to release NO at the site of a wound (J A Bauer et al., Wound Rep. Reg. 6:569-577 (1998)). Alternatively, intense illumination with laser light, e.g., 441 nm light from a HeCd laser, can release NO which is bound to hemoglobin at the site of a wound (Y Vladimirov et al., J. Photochem. Photobiol. B 59:115-122 (2000)).

Additional therapies which can optionally be employed with this embodiment include methods of increasing the bioactivity of NO at the site of a wound by reducing the breakdown of NO. Antioxidants such as glutathione, vitamin E, ascorbic acid, probucol, raxofelast, and related compounds which are known to react with and destroy reactive oxygen species can be administered to the patient. Antioxidants can be administered in any desired form, including as pure substances, in various formulations or combinations, or in the form of commonly available nutritional supplements. Administration of antioxidant therapy can be performed either systemically, e.g., by oral or parenteral administration, or by application at the site of the wound either topically or by injection. Appropriate and effective doses for reducing oxidant stress are known in the art and can be adjusted by the practitioner according to the condition of the patient, such as the level of an oxidant stress-related product or the NOBI. The patient can also be placed on a diet that is rich in natural sources of antioxidants, as are well known in the art. Other forms of dietary treatment involve the reduction of sugar and carbohydrate intake, as well as the reduction of foods that are rich in cholesterol and triglycerides.

In some patients, the bioactivity of NO can be enhanced by the use of antioxidants, which destroy reactive oxygen species before they can react with NO or cause lipid peroxidation. Numerous studies have demonstrated the effectiveness of antioxidant therapy to counteract oxidant stress and to improve NO-dependent endothelial function. For example, alpha tocopherol has been shown to prevent loss of NO-dependent endothelial function in hypercholesterolemia and diabetes mellitus and to improve endothelium-dependent vasodilation in humans (Tomasian, supra). Similar results have been obtained using ascorbic acid, which can scavenge superoxide and inhibit lipid peroxidation (TS Jackson et al., Circ Res 83:916 (1998)). Ascorbic acid also inhibits oxidation of cellular glutathione (Tomasian, supra); glutathione availability helps to maintain NO availability (JA Vita et al., J Clin Invest 101:1408 (1998); K Kugiyama et al., Circulation 97:2299 (1998); A Prasad et al., J Am Coll Cardiol 34:507 (1999)). Physiological concentrations of ascorbic acid have been shown to reverse endothelial dysfunction in patients with congestive heart failure, cigarette smoking, hyperhomocysteinemia, and vasospastic angina. Supraphysiological concentrations of ascorbic acid (obtained by intra-arterial infusion) have demonstrated improved microvascular function in patients with hypercholesterolemia, hypertension, and smoking. (Tomasian, supra). Probucol inhibits LDL oxidation and improves endothelium-dependent vasodilation in animals with experimental hypercholesterolemia (J F Keaney Jr. et al., J Clin Invest 95:2520 (1995)). Flavonoids are also known to inhibit lipid peroxidation and scavenge reactive oxygen species (S A Wiseman et al., Crit Rev Food Sci Nutr 37:705 (1997); D Lairon et al., Curr Opin Lipid 10:23 (1999)). Several enzymes function as antioxidants by degrading reactive oxygen species as part of normal cellular antioxidant defenses. These enzymatic antioxidants include superoxide dismutase, catalase, and glutathione peroxidase. Administration of either the enzymes themselves or genetic vectors encoding their synthesis is expected to increase the bioactivity of NO (Tomasian, supra).

Optionally, the administration of any therapy designed to increase NO production or reduce NO degradation in a subject can be combined with the method described below to monitor the effectiveness of the therapy in enhancing NO levels in the subject. If the subject is found to have a normal NOBI, the preferred treatment does not involve a therapeutic agent designed to increase NO production in the subject. In the case of a subject with normal endothelial function, a different type of therapy can be employed. For example, administration of PDGF (Regranex®, or another PDGF preparation) or KGF can be effective to promote wound healing in the absence of endothelial dysfunction.

The invention can also be used to avoid therapies or diets which may be disadvantageous for certain patients. Any negative influence on the synthesis of NO or its effectiveness in promoting endothelial performance can be minimized through the use of the invention. For example, glucocorticoid drugs are sometimes administered to diabetic patients with LEU for their anti-inflammatory effect. However, glucocorticoids are known to selectively inhibit the expression of iNOS (M W Radomski, R M Palmer, S Moncada, Proc Natl Acad Sci USA 87, 10043 (1990)), and have been shown to decrease the amount of nitrite/nitrate in wound fluid (A E Ulland, J D Shearer, M D Caldwell, J Surg Res 70, 84 (1997)). For patients identified as non-wound healing diabetics, whose NO synthetic capability is expected to be reduced compared with wound healing diabetics, the use of steroids that would further suppress NO levels in the patient is undesirable. Thus, according to one embodiment of the invention, a patient identified as a non-wound healing diabetic is not treated with glucocorticoids or other drugs suspected to reduce NO levels in the patient. In some patients the use of steroids can lead to the formation of steroid ulcerations. Such ulcerations are themselves a condition characterized by chronically impaired wound healing in some patients, and thus are amenable to analysis, monitoring, and treatment according to any of the methods of the present invention. Thus, steroid ulcerations can be related to deficient bioactivity of NO, and the healing ability of a patient with a steroid ulceration can be predicted by analysis of NO-related products. Steroid ulcerations can also be treated using agents which increase NO production, such as L-arginine, hyperbaric oxygen therapy, or therapy involving transfer of an iNOS gene. Alternatively, steroid ulcerations can be treated using antioxidants such as glutathione, vitamin E, ascorbic acid, and related compounds. As another example, the invention can be used to monitor the effects of a subject's diet on endothelial performance. If the subject is found to have endothelial dysfunction, then the subject may be instructed to avoid a diet high in cholesterol or triglycerides.

In a different embodiment, the invention can be used as a method of monitoring the effectiveness of treatment of a condition related to endothelial dysfunction. The method comprising treating a patient using a treatment modality designed to raise the level of NO or reduce oxidant stress, i.e., increase the bioactivity of NO in the patient. The treatment modality is selected from the group consisting of administering an antioxidant, administering a therapeutic agent designed to raise the level of nitric oxide in the patient, administering or providing instructions for a diet, and administering a drug that lowers plasma cholesterol. The method further comprises the step of determining the NOBI in a specimen from the patient as a measure of the effectiveness of the treatment. The method further comprises the step of comparing the NOBI with a threshold that distinguishes whether the patient has endothelial dysfunction. If the patient's NOBI is approximately at or below the threshold value, the effectiveness of the treatment is insufficient to treat endothelial dysfunction. Examples of such therapeutic agents include the L-arginine and antioxidant treatments described above. Following administration of the therapeutic agent, the patient is monitored for effectiveness of the treatment by the method of determining the NOBI in a specimen from the patient, as described above. If the NOBI in the specimen is at or below the threshold value for determining whether the patient has endothelial dysfunction, then the effectiveness of the therapeutic agent is insufficient to treat endothelial dysfunction. In that case, the treatment can be subsequently adjusted, for example by increasing the dose or potency of the therapeutic agent or increasing the period of exposure to the therapeutic agent. In a related embodiment, the method of monitoring the patient is repeated, and the dose or potency of the therapeutic agent, or period of exposure to the therapeutic agent, is again increased. Preferably, in this embodiment the method of monitoring and increasing the dose of the therapeutic agent is increased until the NOBI in a specimen from the patient is above the threshold value. It may be desirable to then maintain the therapy at the most effective dose as long as needed until the patient's endothelial dysfunction has abated.

Still another embodiment is a method for determining if a subject is at risk for developing post-operative wound healing complications. The NOBI of the subject can be determined pre-operatively and a surgeon can use the results to determine if the subject may be at risk for developing post-operative wound healing complications. Post-operative wound healing complications include, for example, non-healing surgical wounds. If a subject is determined to be at risk for developing post-operative wound healing complications, the surgeon can take necessary precautions pre-operatively. Pre-operative precautions include treating the subject by any therapy which is designed to increase NO bioactivity. Such treatments are described above. The surgeon can monitor NOBI and determine when the subject's risk for developing post-operative surgical wound healing complications is reduced.

Yet another embodiment is a kit for determining whether a subject has endothelial dysfunction. The kit comprises one or more reagents for determining either the level of an NO-related product, the level of an oxidant stress-related product, or the NOBI in a specimen from a subject. The reagent or reagents can be those required by any method known in the art for determination of either the level of an NO-related product, the level of an oxidant stress-related product, or the NOBI in a specimen. The kit can also include a set of instructions for using the reagents to carry out the method of determining whether a subject has endothelial dysfunction, as described above. The instruction set provides information in any suitable format (e.g., printed on paper or in electronic format on a diskette, CD-ROM, or by reference to a web site or printed publication) to allow the user to collect a suitable specimen, process the specimen, use the reagent or reagents to determine either the level of an NO-related product, the level of an oxidant stress-related product, or the NOBI in the specimen, and interpret the results obtained, i.e., to compare the results to a threshold which allows the user to determine whether the subject has endothelial dysfunction. In a preferred embodiment, the NO-related product whose level is determined by using the kit is plasma nitrate and the oxidant stress-related product whose level is determined by using the kit is plasma F₂ isoprostane.

Use of NOBI with Other Biomarkers

The NOBI can be used with other biomarkers. Biomarkers are well known in the art and include, for example, cholesterol, LDL, HDL, VLDL, triglycerides, C-reactive protein, and glucose. The NOBI can be charted with a biomarker and used, for example, to assess a risk for a disease. For example, the NOBI can be charted with cholesterol to assess a subject's risk for developing arteriosclerosis.

The above disclosure generally describes the present invention. A more complete understanding can be obtained by reference to the following specific examples, which are provided herein for purposes of illustration only and are not intended to limit the scope of the invention. All patents, patent applications, and references cited in this application are herein incorporated by reference in their entirety.

Screening for Genetic Mutations

Following determination of the NOBI in a sample from the subject, the sample can be screened for genetic mutations that lead to or promote conditions related to endothelial dysfunction. Preferably, genes from the NO synthesis pathway (e.g., iNOS) or degradation pathway (e.g., superoxide dismutase, catalase, and glutathione peroxidase) are screened. Detecting a genetic mutation in a sample can be predictive of a pending endothelial dysfunction, or can be diagnostic of a cause of endothelial dysfunction.

For example, if the NOBI value indicates endothelial dysfunction, a genetic screen can reveal if the root cause of the endothelial dysfunction is a genetic mutation in the NO synthesis or degradation pathways. If the genes in the NO synthesis or degradation pathways are, for example, wild type, then the endothelial dysfunction is likely caused by e.g., excess lipid peroxidation or an insufficient amount of L-arginine in the subject's diet. However, if the genes in the NO synthesis or degradation pathways are, for example, mutant, then the endothelial dysfunction is likely caused by, for example, 1) a mutant iNOS gene, 2) a mutant superoxide dismutase, catalase, or glutathione peroxidase gene, 3) insufficient NO synthesis, 4) excess lipid peroxidation, or 5) a combination of 1, 2, 3, and/or 4.

The NOBI value for a sample from a subject can also indicate that the subject does not have endothelial dysfunction. However, results from a genetic screen of the sample can be predictive of a pending endothelial dysfunction. For example, if the NOBI value indicates that the subject does not have endothelial dysfunction, but the genetic screen shows, for example, a mutation in superoxide dismutase, catalase, or glutathione peroxidase, the subject may experience endothelial dysfunction during periods of oxidant stress.

Treatment for a subject can be tailored to suit the subject and is typically based on the value of NOBI (or two-dimensional analysis of NOBI) and results from the genetic screen.

### EXAMPLE 1

Specimen Collection from Wound Healing and Non-Wound Healing Diabetic Patients

To explore the hypothesis that non-wound healing diabetics have impaired NO activity, the following study of plasma and urinary NO metabolites—nitrate and nitrite—was carried out following a diabetic ulcer wound healing study using Regranex®. The results indicate that the chronic, non-healing LEU diabetic population is characterized by significantly decreased urinary nitrate excretion.

For the clinical study, ten (10) healthy, diabetic patients presenting with a history of one or more diabetic foot ulcers were chosen. All patients had previously received topical ulcer treatment with Regranex® gel under close clinical observation. Half of this group (n=5) experienced complete healing (healed diabetics/HD) of the ulcer by week 20 of observation. The remaining half (n=5) of this group had not experienced complete healing (unhealed diabetics/UHD) of the ulcer by week 20 of observation. Following the completion of Regranex® treatment, the 10 diabetic subjects (HD and UHD) and 10 healthy, non-diabetic controls (C) were enrolled for urine and plasma nitrate/nitrite analysis. Prior to this analysis all subjects were screened with a medical history, physical examination, and baseline hematology, serum, and urine chemistry in order to eliminate subjects with active malignant disease, rheumatic or collagen vascular disease, chronic renal insufficiency, inflammatory bowel disease, alcohol/drug abuse, cellulitis, osteomyelitis and those requiring revascularization surgery. For compliancy reasons, subjects determined to exhibit poor diabetic control were disqualified. Additionally, anyone receiving radiation therapy, systemic corticosteroids, and immunosuppressive or chemotherapeutic agents was disqualified. Informed consent was obtained from all study participants.

Subjects from all groups were brought into the hospital environment after having fasted for a 10-hour period. Fasting urine and plasma samples were obtained from each subject upon admission (Day 1) to provide an indication of the subject's baseline nitrate and nitrite levels. Additionally, routine lab work consisting of chemistry panel, CBC, and urinalysis was obtained from all subjects upon hospital admission. Subjects were confined to the hospital setting for 24 hours, during which time activity level, dietary intake, and other environmental factors were controlled. All subjects were restricted to bed rest with bathroom privileges and consumed the same diet during the 24 hr hospitalization (2,581 Kcals; 124.2 g protein; 5,779 mg arginine; see Table 1). All subjects were required to refrain from smoking and alcohol consumption. Vegetables that usually have a higher nitrate content from fertilizers and nitrate- and nitrite-preserved foods were eliminated from the study diet, which is shown in Table 1 below. Concomitant baseline medications were administered and blood glucose monitoring was performed by the diabetic subjects per their usual home routine. Medication and dietary intake as well as urinary output were recorded and evaluated by the research team during the 24-hour confinement period. At 9 p.m. on the day of confinement, all subjects were required to begin another 10-hour fasting period. Prior to discharge from the hospital setting, the subjects again provided fasting plasma and urine samples (Day 2). Vital signs were monitored daily during confinement and all subjects were evaluated for adverse events prior to discharge. All obtained plasma and urine samples were immediately frozen at −20° C. in preparation for laboratory analysis.

TABLE 1

| | | | Arginine |
|---|---|---|---|
| Research Diet Menu | Calories | Protein (g) | (mg) |
| BREAKFAST | | | |
| egg, 1 | 77 | 6.3 | 377 |
| cereal, ¾ cold | 80 | 3.0 | 240 |
| toast, 1 slice, while | 61 | 2.2 | n/a |
| margarine, 1 tsp. | 45 | 0 | 1 |
| jelly, 1 tbsp. | 50 | 0 | n/a |
| orange juice, 4 oz. | 55 | 0 | 112 |
| milk, 2%, 8 oz. | 121 | 8.1 | 294 |
| LUNCH | | | |
| hamburger, 3.5 oz. | 274 | 26.6 | 1615 |
| bun | 122 | 4.4 | n/a |
| ½ sliced tomato | 15 | .5 | 8 |
| mayonnaise, 1 tbsp. | 100 | 0 | 10 |
| corn chips, 1 oz. | 153 | 1.9 | 92 |
| mixed fruit cup | 80 | 0 | n/a |
| milk, 2%, 8 oz. | 121 | 8.1 | 294 |
| DINNER | | | |
| baked chicken breast, 3.5 oz. | 222 | 29.0 | 1811 |
| rice, white, ½ c. | 133 | 4.9 | 342 |
| apples, canned, 1/2 c. | 68 | 0 | 6 |
| dinner roll | 85 | 2.4 | n/a |
| margarine, 1 tsp. | 45 | 0 | 1 |
| cantaloupe cubes, 1/2 c. | 28 | 1.4 | n/a |
| diet pudding, 1/2 c. | 250 | 6.2 | n/a |
| milk, 2%, 8 oz. | 121 | 8.1 | 294 |

### EXAMPLE 2

Determination of Urine and Plasma Nitrate and Nitrite Concentrations in Wound Healing and Non-Wound Healing Diabetic Patients

Specimens of urine and blood were obtained from the wound healing and non-wound healing diabetic subjects as described in Example 1.

Urine and plasma samples were fluorometrically assayed for nitrite and nitrate levels using a commercial kit (Cayman Chemical, Ann Arbor, Mich.) according to the manufacturer's instructions. The method used is that described by Gilliam et al. (Anal. Biochem. 212, 359 (1993)). Blood was collected in a glass tube, centrifuged, and the plasma collected and frozen until assay. The samples were thawed, vortexed and filtered with a 10 kDa size exclusion filter (Millipore, Bedford, Mass.). For the determination of nitrate, nitrate reductase and NADP was added and allowed to incubate at room temperature for two hours. Following incubation 2,3-diaminonapthalene followed by NaOH was added and the fluorescence determined with a fluorimeter using excitation at 365 nm and emission at 405 nm. Nitrite concentration was determined using the same method with the exception that nitrate reduction steps were omitted. Urine was processed in a similar fashion except that filtration was omitted. All samples were assayed in triplicate. Concentration in patient samples (micromoles per liter) was determined by comparison to standard nitrate and nitrite solutions. One-way ANOVA with Tukey-Kramer post test (Ludbrook, Clin. Exp. Pharmacol. Physiol. 18, 379 (1991)) was performed using GraphPad InStat software, version 4.10 for Windows 98. P-values <0.05 were considered significant.

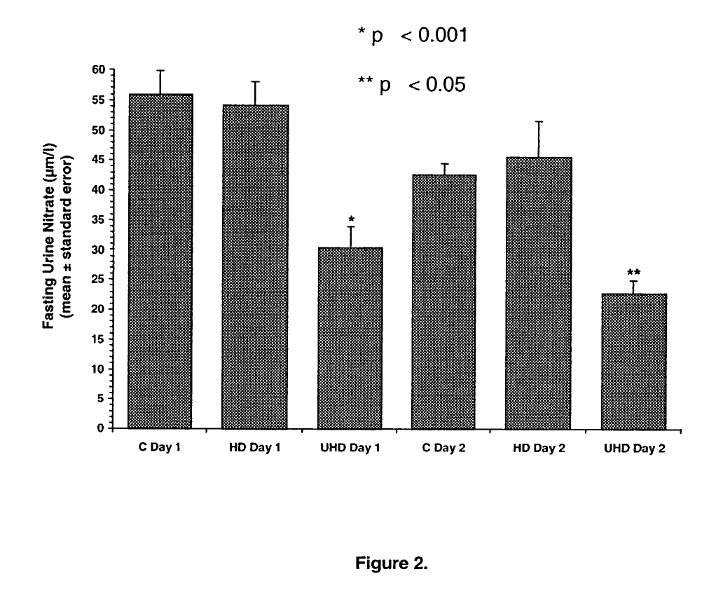

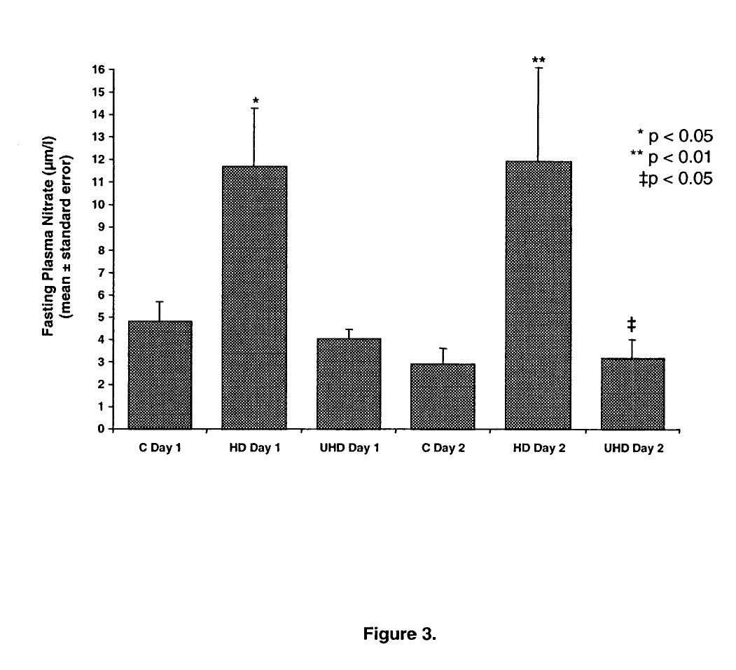

On Day-1 fasting urine nitrate levels (micromoles/1±S.E.) for groups C and HD (55.88±4.49 and 54.14±3.32, respectively) were not significantly different (FIG. 2). However, group UHD fasting urine nitrate levels (30.35±3.61) were significantly lower than groups C (p<0.001) or HD (p<0.01). Day-2 fasting urine nitrate levels for groups C and HD were lower (42.60±1.92 and 45.57±5.10, respectively) but again not significantly different. Similarly, group UHD fasting urine nitrate levels (22.74±3.13) were lower than Day-1 values and were again significantly lower than either group C (p<0.05) or HD (p<0.05) [Table 2]. Day-1 fasting plasma nitrate levels (micromoles/1±S.E.) for group C (4.80±0.85) and group UHD (4.05±0.37) were not significantly different (FIG. 3). Group HD (11.71±2.08), however, was higher than UHD, but only significantly higher than C (p<0.05). Day-2 fasting plasma nitrate levels were slightly lower for groups C (2.92±0.37) and UHD (3.16±0.61), but as before these were not significantly different. However, Group HD (11.94±4.46) was now significantly higher that either group C (p<0.01) or UHD (p<0.05). Urine and plasma nitrate levels were approximately 100 times greater than nitrite and were occasionally undetectable by this methodology. For these reasons urine and plasma nitrite levels are not reported.

TABLE 2
Fasting Urine and Plasma Nitrate Values

| | DAY 1 | | | DAY 2 | | |
|---|---|---|---|---|---|---|
| | C | HD | UHD | C | HD | UHD |
| | N = 10 | N = 5 | N = 5 | N = 10 | N = 5 | N = 5 |
| Fasting Urine | 55.88 ± | 54.14 ± | 30.35 ± | 42.60 ± | 45.57 ± | 22.74 ± |
| Nitrate* (μm/l) | 4.49 | 3.32 | 3.61 | 1.92 | 5.10 | 3.13 |
| P †, ‡ | | †NS | †.001 | | ‡NS | ‡.05 |
| Fasting Plasma | 4.80 ± | 11.71 ± | 4.05 ± | 2.92 ± | 11.94 ± | 3.16 ± |
| Nitrate* (μm/l) | 0.85 | 2.08 | 0.37 | 0.37 | 4.46 | 0.61 |
| P †, ‡, § | | †0.05 | †NS | | ‡0.01 | ‡NS |
| | | | | | | §0.05 |

LEGEND
*Mean ± standard error
§Compared to HD, Day 2
C Control Group
P P Value
†Compared to Controls, Day 1
‡Compared to Controls, Day 2
HD Healed Diabetics
UHD Unhealed Diabetics

### EXAMPLE 3

Determination of NO Bioactivity Index for Diabetic Patients.

The levels of plasma nitrate and plasma isoprostane are determined for a group of diabetic patients. Plasma nitrate levels are determined as described in Example 2 following fasting and administration of a low nitrate diet as described in Example 1. 8-Isoprostane (8-epi prostaglandin F 2 alpha) is determined according to the instructions of the Cayman Chemical 8-Isoprostane EIA Kit (Cat. No. 516351). The results are displayed in FIG. 5.

A linear relationship is observed for a plot of plasma isoprostane on the horizontal axis and plasma nitrate on the vertical axis in the case of control patients and wound healing diabetic patients. The slope of the best fit line by linear regression analysis is 26.7±1.7 SEM micromoles nitrate per nanomole isoprostane. The mean NOBI using plasma nitrate and plasma isoprostane for the group of controls and wound healing diabetics is therefore 26.7 micromoles nitrate per nanomole isoprostane. Non-wound healing diabetic patients have an NOBI lower than 26.7, such as about 4.1 micromoles nitrate per nanomole isoprostane.

1. A method of determining whether a subject has endothelial dysfunction, comprising the step of: comparing the nitric oxide bioactivity index in a specimen from the subject with a threshold value, wherein the nitric oxide bioactivity index is defined as the level of a nitric oxide-related product in the specimen divided by the level of an oxidant stress related product in the specimen, wherein if the nitric oxide bioactivity index is above the threshold value the subject does not have endothelial dysfunction, and if the nitric oxide bioactivity index is approximately at or below the threshold value the subject has endothelial dysfunction.

2. A method of determining whether a subject has endothelial dysfunction, comprising the step of: comparing the nitric oxide bioactivity index in a specimen from the subject with a threshold value, wherein the nitric oxide bioactivity index is defined as the level of an oxidant stress related product in the specimen divided by the level of an nitric oxide-related product in the specimen, wherein if the nitric oxide bioactivity index is below the threshold value the subject does not have endothelial dysfunction, and if the nitric oxide bioactivity index is approximately at or above the threshold value the subject has endothelial dysfunction.

3. The method of claim 1 or 2, further comprising the step of: determining the level of a nitric oxide-related product in the specimen.

4. The method of claim 1 or 2, further comprising the step of: determining the level of an oxidant stress-related product in the specimen.

5. The method of claim 1, further comprising the step of: dividing the level of a nitric oxide-related product in the specimen by the level of an oxidant stress-related product in the specimen to obtain the nitric oxide bioactivity index.

6. The method of claim 2, further comprising the step of: dividing the level of an oxidant stress-related product in the specimen by the level of a nitric oxide-related product in the specimen to obtain the nitric oxide bioactivity index.

7. The method of claim 1 or 2, wherein the specimen is urine, blood, or tissue.

8. The method of claim 1 or 2, wherein the subject is a human.

9. The method of claim 1 or 2, wherein the subject is suspected of having a medical condition related to endothelial dysfunction.

10. The method of claim 9, wherein the condition is selected from the group consisting of diabetes, preclinical diabetes, hypertension, atherosclerosis, atherosclerotic peripheral vascular disease, chronic diabetic ulcer, venous stasis ulcer, decubitus ulcer, steroid-dependent ulcer, chronic venous insufficiency, sickle cell disease, trauma, chronic non-healing burn injury, chronic non-healing surgical wound, chronic osteomyelitis, erectile dysfunction, postmenopausal state, preeclampsia, cigarette smoking, acute respiratory distress syndrome (ARDS), radiation injury, spinal cord injury, malnutrition, sepsis, chronic soft tissue infection, vitamin deficiency, osteoporosis, post-operative surgical wound, and old age.

11. The method of claim 1 or 2, wherein the nitric oxide-related product is selected from the group consisting of nitrate, nitrite, nitric oxide, L-citrulline, cGMP, peroxynitrite, 3-nitrotyrosine, and L-dimethylarginine.

12. The method of claim 1 or 2, wherein the oxidant stress-related product is selected from the group consisting of an isoprostane, malondialdehyde, a conjugated diene, a thiobarbituric acid reactive substance, 4-hydroxynonenal, an oxidized low density lipoprotein, and an advanced glycation end product.

13. The method of claim 1 or 2, further comprising the step of collecting the specimen.

14. The method of claim 11, wherein the nitric oxide-related product is nitrate or nitrite and the specimen is collected following a fasting period of at least 6 hours.

15. The method of claim 14, wherein the fasting period is from 6 to 10 hours.

16. The method of claim 15, further comprising the step of: administering to the subject for at least 6 hours immediately prior to the fasting period a diet with an intake of nitrate below 900 mg/kg body weight/day and an intake of nitrite below 9 mg/kg body weight/day.

17. The method of claim 16, wherein the diet is administered for a period of from 6 to 24 hours in duration.

18. The method of claim 5, wherein the specimen is blood, the nitric oxide-related product is nitrate, the oxidant stress-related product is isoprostane, and the threshold value of the nitric oxide bioactivity index is between 10 and 40 micromoles nitrate per nanomole isoprostane.

19. The method of claim 18, wherein the threshold value is between 20 and 30 micromoles nitrate per nanomole isoprostane.

20. The method of claim 19, wherein the threshold value is between 23 and 27 micromoles nitrate per nanomole isoprostane.

21. The method of claim 20, wherein the threshold value is about 25 micromoles nitrate per nanomole isoprostane.

22. A method of treating a subject having a condition related to endothelial dysfunction, comprising the steps of: determining the nitric oxide bioactivity index using a specimen from the subject according to the method of claim 1 or 2, and treating the subject according to the nitric oxide bioactivity index.

23. The method of claim 22, wherein the condition is selected from the group consisting of diabetes, preclinical diabetes, hypertension, atherosclerosis, atherosclerotic peripheral vascular disease, venous stasis ulcer, chronic diabetic ulcer, decubitus ulcer, steroid-dependent ulcer, chronic venous insufficiency, sickle cell disease, trauma, chronic non-healing burn injury, chronic non-healing surgical wound, chronic osteomyelitis, erectile dysfunction, postmenopausal state, preeclampsia, cigarette smoking, acute respiratory distress syndrome (ARDS), radiation injury, spinal cord injury, malnutrition, sepsis, chronic soft tissue infection, vitamin deficiency, osteoporosis, post-operative surgical wound, and old age.

24. The method of claim 22, wherein the step of treating comprises a method selected from the group consisting of (a) administering L-arginine to the subject, (b) administering a nitric oxide releasing agent to the subject, (c) administering an antioxidant to the subject, (d) administering to the subject a gene transfer vector comprising a polynucleotide encoding an iNOS enzyme, (e) performing hyperbaric oxygen therapy on the subject, (f) administering to the subject a drug that lowers plasma cholesterol or triglycerides, and (g) administering a diet to the subject or instructing the subject to adhere to a diet.

25. The method of claim 24, wherein the subject is administered an antioxidant selected from the group consisting of ascorbic acid, alpha tocopherol, raxofelast, probucol, a flavonoid, and an enzymatic antioxidant.

26. The method of claim 25, wherein the enzymatic antioxidant is selected from the group consisting of superoxide dismutase, catalase, and glutathione peroxidase.

27. The method of claim 24, wherein the diet includes one or more foods comprising a natural antioxidant.

28. The method of claim 24, wherein the diet includes one or more dietary supplements comprising an antioxidant.

29. The method of claim 24, wherein the diet is low in cholesterol or triglycerides.

30. The method of claim 24, wherein the drug that lowers cholesterol or triglycerides is a statin.

31. A method of monitoring the effectiveness of treatment of a condition related to endothelial dysfunction, comprising the steps of: (a) treating the patient using a treatment modality selected from the group consisting of (i) administering an antioxidant, (ii) administering a therapeutic agent designed to raise the level of nitric oxide in the patient, (iii) administering or providing instructions for a diet, and (iv) administering a drug that lowers plasma cholesterol; (b) determining the nitric oxide bioactivity index in a specimen from the patient as a measure of the effectiveness of the treatment, wherein the nitric oxide bioactivity index is defined as the level of a nitric oxide-related product in the specimen divided by the level of an oxidant stress related product in the specimen; and (c) comparing the nitric oxide bioactivity index with a threshold that distinguishes whether the patient has endothelial dysfunction, wherein if the nitric oxide bioactivity index is above the threshold value the effectiveness of treatment is sufficient to treat endothelial dysfunction, and if the nitric oxide bioactivity index is approximately at or below the threshold value the effectiveness of the treatment is insufficient to treat endothelial dysfunction.

32. The method of claim 31, wherein the nitric oxide-related product is selected from the group consisting of nitrate, nitrite, nitric oxide, L-citrulline, cGMP, peroxynitrite, 3-nitrotyrosine, or L-dimethylarginine.

33. The method of claim 31, wherein the oxidant stress-related product is selected from the group consisting of an isoprostane, malondialdehyde, a conjugated diene, a thiobarbituric acid reactive substance, 4-hydroxynonenal, and an oxidized low density lipoprotein.

34. The method of claim 31, further comprising the step of, prior to administering a therapeutic agent to the patient: identifying the patient as having endothelial dysfunction by the method of claim 1.

35. The method of claim 31, further comprising the step of: (d) adjusting the treatment according to the nitric oxide bioactivity index, wherein if the level is at or below the threshold value the administration of the therapeutic agent is increased, and if the level is above the threshold the administration of the therapeutic agent is not increased.

36. The method of claim 35, wherein if the nitric oxide bioactivity index following administration of the therapeutic agent is at or below the threshold level, the method further comprises the step of: (e) repeating steps (a) through (d) until the nitric oxide bioactivity index in a specimen from the patient is above the threshold level.

37. A kit for determining whether a subject has endothelial dysfunction, comprising either (1) one or more reagents for determining the level of a nitric oxide-related product in a specimen from the subject, (2) one or more reagents for determining the level of an oxidant stress-related product in a specimen from the subject, or (3) both (1) and (2).

38. The kit of claim 37, wherein the specimen is urine, blood, or tissue.

39. The kit of claim 37, wherein the nitric oxide-related product is selected from the group consisting of nitrate, nitrite, nitric oxide, L-citrulline, cGMP, peroxynitrite, 3-nitrotyrosine, or L-dimethylarginine.

40. The kit of claim 37, wherein the oxidant stress-related product is selected from the group consisting of an isoprostane, malondialdehyde, a conjugated diene, a thiobarbituric acid reactive substance, 4-hydroxynonenal, and an oxidized low density lipoprotein.

41. The kit of claim 37, wherein the nitric oxide-related product is nitrate and the oxidant stress-related product is an isoprostane.

42. The kit of claim 37 further comprising instructions.

43. The method of claim 1 or 2 further comprising the step of comparing the nitric oxide bioactivity index with a biomarker.

44. The method of claim 1 or 2, further comprising the step of screening for a genetic mutation that leads to or promotes a condition related to endothelial dysfunction.

45. The method of claim 44, further comprising the step of detecting a genetic mutation that leads to or promotes a condition related to endothelial dysfunction.

46. The method of claim 44, wherein the genetic mutation is in a gene from the NO synthesis pathway.

47. The method of claim 46, wherein the gene from the NO synthesis pathway is iNOS.

48. The method of claim 44, wherein the genetic mutation is a genetic mutation in a gene from the NO degradation pathway.

49. The method of claim 48, wherein the gene from the NO degradation pathway is selected from the group consisting of superoxide dismutase, catalase, and glutathione peroxidase.

50. The method of claim 44, wherein the condition is selected from the group consisting of diabetes, preclinical diabetes, hypertension, atherosclerosis, atherosclerotic peripheral vascular disease, chronic diabetic ulcer, venous stasis ulcer, decubitus ulcer, steroid-dependent ulcer, chronic venous insufficiency, sickle cell disease, trauma, chronic non-healing burn injury, chronic non-healing surgical wound, chronic osteomyelitis, erectile dysfunction, postmenopausal state, preeclampsia, cigarette smoking, acute respiratory distress syndrome (ARDS), radiation injury, spinal cord injury, malnutrition, sepsis, chronic soft tissue infection, vitamin deficiency, osteoporosis, post-operative surgical wound, and old age.
